(19) United States
(12) Patent Application Publication
(10) Pub. No.: US 2016/0022923 A1
(43) Pub. Date: Jan. 28, 2016

(54) VEIN STABILIZER DEVICE

(71) Applicant: Russell Curtis, Argos, IN (US)

(72) Inventor: Russell Curtis, Argos, IN (US)

(21) Appl. No.: 14/811,737

(22) Filed: Jul. 28, 2015

Related U.S. Application Data
(60) Provisional application No. 62/029,938, filed on Jul. 28, 2014.

(51) Int. Cl. A61M 5/32

(52) U.S. Cl. CPC A61M 5/3287

(57) ABSTRACT

A vein stabilizer device for holding a vein in place and receiving a needle to be inserted therein. The vein stabilizer device includes a base tube defined by a hollow tube body having two receiving flanges disposed on opposing sides of its outer surface and two detachable extension arms which are each defined by an elongated member having a proximal end attached to one of the receiving flanges, an angled bend, and distal end which includes a removable tip. In operation, a user slides a syringe into the base tube with the needle facing towards and extending almost to the removable tips of the opposing extension arms. The removable tips are then placed against the skin on either side of the target vein, allowing the needle to be inserted into a target vein that is held between the removable tips.

### CROSS REFERENCE TO RELATED APPLICATIONS

This application claims the benefit of and incorporates by reference co-pending U.S. provisional patent application serial number 62/029,938 filed Jul. 28, 2014.

### BACKGROUND OF THE INVENTION

1. Field of the Invention

This invention relates generally to medical procedure devices and, more particularly, to a vein stabilizer device having a tube member and a plurality of clamp members.

2. Description of the Prior Art

The use of hypodermic needles with syringes to inject substances into a patient's body is well known. A problem which still exists, however, is caused by the tendency of veins to move, particularly side to side, when medical personnel or other users of such needles and syringes attempt to penetrate the vein with the tip of a needle. This often results in undesirable consequences, such as the need for additional personnel to hold the vein in place or a patient having to be pricked repeatedly in order to properly receive their injection and the delivery of fluids and/or medication. Thus, there remains a need for a vein stabilizer device which holds and prevents veins from rolling while a user is inserting a needle therein. It would be helpful if such a vein stabilizer device was adapted to additionally hold the syringe with the needle to be inserted to form a single injection assembly. It would be additionally desirable for such a vein stabilizer device to include two detachable metal arms with rubber tips to enable different sizes of syringes and needles to be used.

The Applicant's invention described herein provides for a vein stabilizer device adapted to allow a single user to prevent a target vein from rolling or otherwise moving from side to side while a user attempts to insert a needle therein. The primary components in Applicant's vein stabilizer device are a base tube and two extension arms, each having stabilizing tips. When in operation, the vein stabilizer device enables the holding of a vein in place while a hypodermic needle is inserted therein. As a result, many of the limitations imposed by prior art structures are removed.

### SUMMARY OF THE INVENTION

A vein stabilizer device for holding a vein in place and receiving a needle to be inserted therein. The vein stabilizer device includes a base tube defined by a hollow tube body having two receiving flanges disposed on opposing sides of its outer surface and two detachable extension arms which are each defined by an elongated member having a proximal end attached to one of the receiving flanges, an angled bend, and distal end which includes a removable tip. In operation, a user slides a syringe into the base tube with the needle facing towards and extending almost to the removable tips of the opposing extension arms. The removable tips are then placed against the skin on either side of the target vein, allowing the needle to be inserted into a target vein that is held between the removable tips.

It is an object of this invention to provide a vein stabilizer device which holds and prevents veins from rolling while a user is inserting a needle therein.

It is another object of this invention to provide a vein stabilizer device adapted to additionally hold the syringe with the needle to be inserted to form a single injection assembly.

It is yet another object of this invention to provide a vein stabilizer device to include two detachable metal arms with rubber tips to enable different sizes of syringes and needles to be used.

These and other objects will be apparent to one of skill in the art.

### BRIEF DESCRIPTION OF THE DRAWINGS

FIG. 1 is a side perspective view of the base tube and extension arm of a vein stabilizer device built in accordance with the present invention.

FIG. 2 is a top plan view of a vein stabilizer device built in accordance with the present invention.

FIG. 3 is a side perspective view of an assembled vein stabilizer device built in accordance with the present invention.

### DETAILED DESCRIPTION OF THE INVENTION

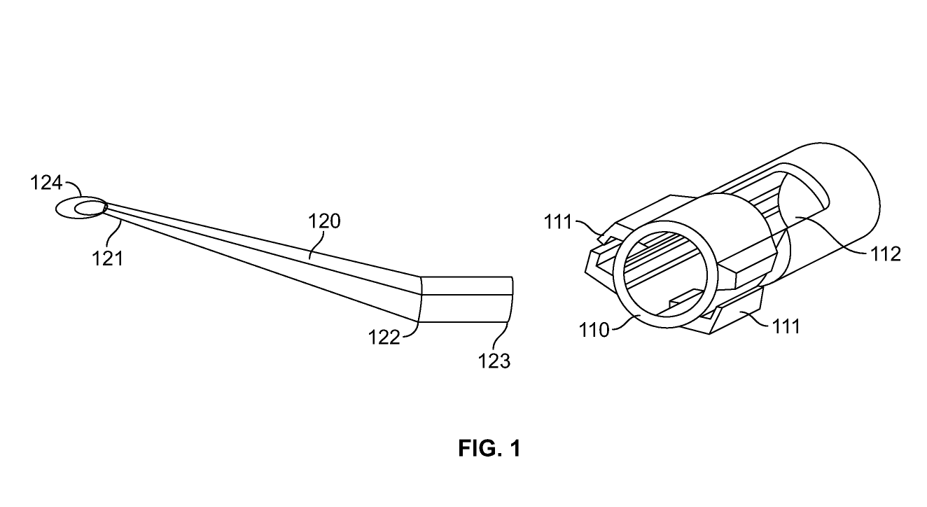

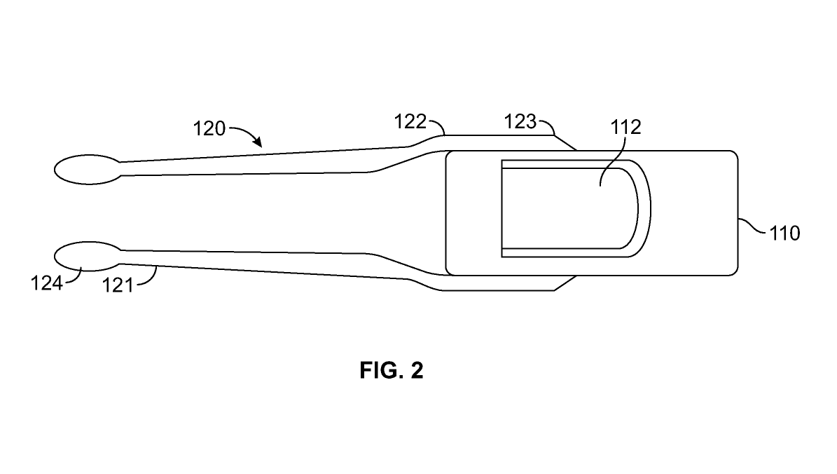

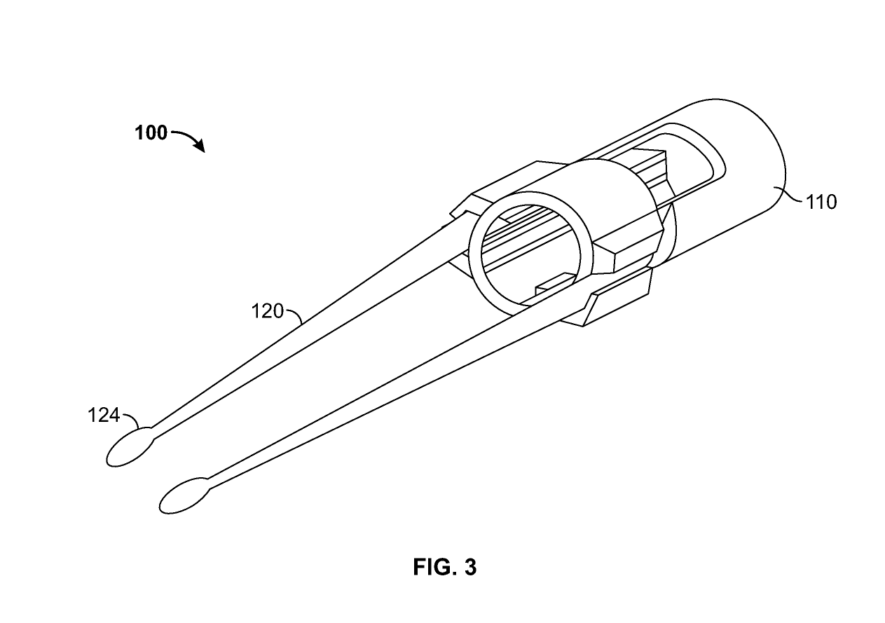

Referring now to the drawings and in particular FIGS. 1, 2 and 3, a vein stabilizer device 100 is shown having a base tube 110 and two detachable extension arms 120. The base tube 110 is defined by a hollow tube body having two receiving flanges 111 disposed on opposing sides of its outer surface. The base tube 110 is sized such that a conventional syringe and hypodermic needle can be passed through its hollow interior. A thumb hole 112 is disposed on the base tube 110, sized to receive the thumb of a user, so that a syringe in place in the hollow interior of the base tube 110 can be manually held by a user. In the preferred embodiment, the base tube 110 is 3 centimeters(“cm”) long, has an interior diameter of 1.5 cm, the receiving flanges are 1 cm long, and the thumb hole 112 is 1.5 cm long. In addition, in the preferred embodiment, the base tube is constructed out of clear plastic.

The extension arms 120 are each defined by an elongated member having a distal end 121, an angled bend 122, and a proximal end 122. At the distal end 121 is a removable tip 124, defined as silicon in the preferred embodiment. The removable tip 124 covers the distal end 121, providing a comfortable surface for contacting a user's skin. The proximal end 123 engages and attaches to one of the receiving flanges 111 on the base tube 110, enabling the extension arm 120 to be attached to the base tube 110. In the preferred embodiment, the extension arms 120 each measure 5 cm total, with the section from the proximal end 123 to the angled bend 122 measuring 1 cm. In addition, in the preferred embodiment, the extension arms are constructed out of metal

In operation, a user slides a syringe into the base tube with the needle facing towards and extending almost to the removable tips 124 of the opposing extension arms 120. The removable tips 124 are placed against the skin, on either side of the target vein, providing a barrier that prevents it from moving to one side or the other. The needle can then be inserted into the target vein as it is held in place.

It is contemplated that the extension arms 120 can be constructed in various lengths, enabling the vein stabilizer device 100 to be customized for various lengths of syringes and needles due to their detachable nature.

It is further contemplated that the vein stabilizer device 100 can be constructed to be utilized with a plurality of needle gauges, including 21, 23, and 25.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A vein stabilizer device, comprising:
a base tube is defined by a hollow body with an exterior surface and sized such to allow a conventional syringe and hypodermic needle can be passed through the hollow body, wherein said base tube includes at least one engaging section; and
at least one extension arm defining an elongated member having a distal end and a proximal end, wherein said proximal end is configured to removably attach to said engaging section of said base tube so as to allow the distal end to rigidly extend laterally from the base tube.

2. The vein stabilizer device of claim 1, additionally comprising a removable tip integral with said distal end, wherein said removable tip is constructed of a non-slip material.

3. The vein stabilizer device of claim 1, wherein said at least one extension arm includes an angled bend section disposed between the proximal end and distal end.

4. The vein stabilizer device of claim 1, wherein said base tube includes an integral thumb hole sized to receive a thumb of a user, thereby enabling the user to hold a syringe in place in the hollow body.

5. The vein stabilizer device of claim 1, wherein said engaging section is disposed on the exterior surface.

6. The vein stabilizer device of claim 1, wherein said base tube includes two engaging sections, each disposed on opposing sides of the exterior surface of the base tube.

7. The vein stabilizer device of claim 1, wherein said engaging section defines a receiving flange.

8. The vein stabilizer device of claim 7, wherein said base tube includes two receiving flanges, each disposed on opposing sides of the exterior surface of the base tube.

9. A vein stabilizer device, comprising:
a base tube is defined by a hollow body with an exterior surface and sized such to allow a conventional syringe and hypodermic needle can be passed through the hollow body, wherein said base tube includes two receiving flanges which are disposed on opposing sides of the exterior surface of the base tube; and
two extension arms, each defining an elongated member having a distal end and a proximal end, as well as an angled bend disposed between the distal and proximal end, wherein said proximal end of each extension arm is configured to removably attach to one of said engaging sections of said base tube so as to allow the distal end to rigidly extend laterally from the base tube.

10. The vein stabilizer device of claim 9, wherein said engaging section defines a receiving flange.

11. The vein stabilizer device of claim 9, wherein said base tube includes an integral thumb hole sized to receive a thumb of a user, thereby enabling the user to hold a syringe in place in the hollow body.

12. The vein stabilizer device of claim 9, additionally comprising a removable tip integral with said distal end, wherein said removable tip is constructed of a non-slip material.
